(19) United States
(12) Patent Application Publication
(10) Pub. No.: US 2010/0056879 A1
(43) Pub. Date: Mar. 4, 2010

(54) Method for maintaining homeostasis in a health care setting

(75) Inventors: Jay S. Greenspan, Merion Station, PA (US); Thomas L. Miller, Wilmington, DE (US); Thomas H. Shaffer, Chadds Ford, PA (US)

Correspondence Address: DRINKER BIDDLE & REATH;ATTN: INTELLECTUAL PROPERTY GROUP, ONE LOGAN SQUARE, 18TH AND CHERRY STREETS, PHILADELPHIA, PA, 19103-6996, US

(73) Assignee: The Nemours Foundation, Jacksonville, FL (US)

(21) Appl. No.: 12/231,461

(22) Filed: Sep. 3, 2008

(51) Int. Cl. A61B 5/02; G08B 23/00

(52) U.S. Cl. 600/301; 340/573.1

(57) ABSTRACT

A method for maintaining homeostasis for a patient in a health care setting includes the steps of monitoring a physiological parameter and comparing the parameter against an expected parameter range. When it is determined that a difference between the monitored physiological parameter and the expected parameter range presents a patient condition requiring a corrective measure, an alarm warning is presented. The alarm warning has a signature characteristic distinguishing it from other alarm warnings. The alarm warning may be musical and distinguishable based on genre, tempo, loudness, or pitch or, alternatively may be visual. The alarm warning may be adapted to distinguish one patient from other patients. Alternatively, the alarm warning may distinguish differing levels of severity associated with a patient condition of a particular patient or to distinguish between multiple conditions suffered by the same patient.

### FIELD OF THE INVENTION

The present invention relates generally to health care settings such as an intensive care, hospital care or home care setting and, more particularly to a method and apparatus for maintaining homeostasis of a patient of the health care setting.

### BACKGROUND OF THE INVENTION

Many patients, including neonates, children and adults in intensive care, hospital care or home care settings suffer from conditions (e.g., apnea, cardiac abnormalities and thermal instability) that require monitoring of the patient and application of a corrective measure (e.g., a waking stimulus such as a nudge applied to an apnea patient) during an episode of the condition. These conditions can lead to life-threatening consequences associated with cardiopulmonary deterioration or respiratory interruption. Apnea, for example, is a condition causing the respiration to halt and can lead to death by auto-asphyxiation during a severe episode.

There are known devices that monitor respiratory, cardiac and/or thermal parameters for patients suffering from the above conditions. Known apnea monitoring devices include devices that measure respiratory gas flows (e.g., end-tidal CO₂) of the patient, as well as devices that use pulse oximetry and/or thoraco-abdominal motion to monitor patient respiration. These devices can be cumbersome, if not invasive (e.g., CO₂ monitoring devices requiring a firm-fitting mask or endotracheal intubation).

Known devices generate an alarm when a monitored physiological parameter is outside of a normal range for the parameter. The information associated with the alarms of conventional devices is limited to indicating that an episode of the condition being monitored has been sensed. The conventional devices, therefore, are not adapted to indicate the severity of the particular condition. The alarm feature of conventional devices are also not adapted to identify and distinguish one patient from a plurality of patients in a health care settings.

What is needed is a device that monitors a respiratory, cardiac and/or thermal parameter for a patient condition (e.g., apnea) that requires a corrective measure (e.g., a waking stimulus) and generates an alarm warning having a signature feature when a corrective measure is required. The signature feature could be used to distinguish the alarm warning from other alarm warnings that might be generated at a health care setting, thereby ensuring that the appropriate corrective measure is applied to the particular patient suffering from the condition. Such a feature could be used, for example, to distinguish the alarm warning for a given patient from other alarm warnings associated with the same patient (e.g., to indicate different patient conditions or to indicate differing severity for a particular condition) or to distinguish the alarm warning(s) of one patient from those of another patient of the health care setting.

### SUMMARY OF THE INVENTION

According to the present invention, a method for maintaining homeostasis for a patient in a health care setting such as an intensive care, hospital care or home care setting, is provided. The method includes the step of monitoring the patient for at least one physiological parameter. The monitored physiological parameter could include a respiratory parameter, a cardiac parameter or a thermal parameter. According to one embodiment, the patient suffers from sleep apnea and is monitored for a respiratory parameter. The monitored physiological parameter is then compared against an expected parameter range for the patient. When it is determined that a difference between the monitored physiological parameter and the expected parameter range presents a patient condition requiring a corrective measure, an alarm warning is presented. The alarm warning of the present invention has a signature characteristic that is adapted to distinguish the alarm warning from other alarm warnings.

According to one embodiment, the alarm warning is audible and musical. The signature characteristic associated with the audible warning is based on a property of the music such as genre, tempo, loudness, pitch, for example. The alarm warning, however, could embody other forms such as visual, for example.

According to one embodiment, the health care setting has a plurality of patients. The signature characteristic of the alarm warning is adapted to distinguish the alarm warning(s) of a given patient from the alarm warnings associated with other patients.

According to one embodiment, the alarm warning is one of a plurality of warnings associated with a given patient. The plurality of warnings could identify varying levels of severity for a particular patient condition suffered by the patient. Alternatively, the plurality of alarm warnings could be used to distinguish between multiple patient conditions of differing type suffered by the same patient.

According to one embodiment, the method further includes the step of applying a corrective measure to the patient. The corrective measure could be applied indirectly, such as by a stimulus device located near the patient, or, alternatively, could be applied directly by a caregiver of the health care setting.

As used herein, the term “homeostasis” as applied to a patient of a health care setting refers to a relatively stable state of equilibrium for the patient.

As used herein, the term “health care setting” includes intensive care facilities, hospitals, and home care environments.

### BRIEF DESCRIPTION OF THE DRAWINGS

For the purpose of illustrating the invention, the drawings show a form of the invention that is presently preferred. However, it should be understood that this invention is not limited to the precise arrangements and instrumentalities shown.

FIG. 1 is a schematic illustration of an apparatus for maintaining homeostasis for a patient in a health care setting according to an exemplary embodiment of the invention.

FIGS. 2 through 5 are flow charts illustrating methods for maintaining homeostasis for a patient according to exemplary embodiments of the invention.

### DESCRIPTION OF THE INVENTION

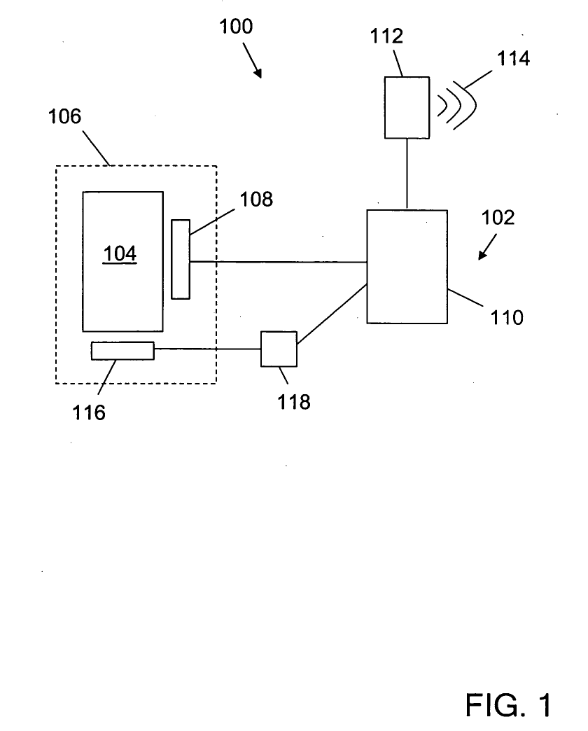

Referring to the drawings where like numerals identify like elements, there is illustrated schematically in FIG. 1 a health care setting 100 (e.g., an intensive care, hospital care or home care setting) having an apparatus 102 for maintaining homeostasis for a patient 104. The apparatus 102 is adapted to monitor the patient for a condition (e.g., apnea) that requires the application of a corrective measure (e.g., a waking stimulus) and to generate an alarm warning when an episode of the condition is sensed to indicate that the application of a corrective measure is required. As described below in greater detail, the alarm warning of the present invention has a signature characteristic that serves to distinguish the alarm warning from alarm warnings of other patients of the health care setting or from other warnings associated with the given patient.

In the depicted care setting 100, the patient 104 is located within a controlled environment 106. Such a patient in a controlled environment could, for example, be a neonate located in an incubator of a neonatal intensive care facility. It should be understood, however, that the invention is not so limited and has application to other patients (e.g., children, adults) in other care settings (e.g., hospital care or home care settings) and to situations in which the patient is not contained within a controlled environment of the care setting.

The apparatus 102 includes one or more monitoring devices 108 each arranged to measure a physiological parameter of the patient 104. Although the invention could have other application, patients suffering from apnea, cardiac abnormalities and thermal instability are particularly contemplated. The physiological parameters associated with such patients, therefore, to be measured by the monitoring devices 108 include respiratory, cardiac and thermal parameters. For example, respiration parameters (e.g., for apnea) could be monitored by end-tidal CO₂, pulse oximetry and/or thoraco-abdominal motion; cardiac parameters could be monitored by EKG signals; and thermal conditions could be monitored by thermisters/thermocouples or hygrometers. Preferably, the monitoring devices 108 utilize less invasive technologies to the extent possible. For example, known monitoring devices for monitoring sleep respiration in clinical and home settings include those that monitor carbon dioxide via nasal tubing and those that monitor thoraco-abdominal motion via elastic respiratory bands. With each expired breath, the fraction of CO₂ in the gas outside the oral and nasal cavities rises, and then falls, with inspiration. Therefore, a CO₂ sensor can monitor the inspiratory and expiratory phases of breathing, and automatically provide data for simple calculations of respiratory rate and time constants. A CO₂ sensor can also detect subtle abnormalities by changes in expired CO₂ peaks. Devices for monitoring respiratory, cardiac and thermal parameters are well known and, therefore, no further description is required.

The apparatus 102 includes a central processing unit (CPU) 110. The CPU 110 receives data from the monitoring devices regarding the physiological parameters measured by the devices. The CPU 110 uses the data, preferably through an algorithm, to determine when a condition of the patient 104 requires the application of a corrective measure such a waking stimulus for a patient suffering from sleep apnea. According to one embodiment, the feedback signal of CO₂ from the monitoring devices 108 can be compared to a set point established by a caregiver (i.e., PCO₂=45 mmHg). If the sensed PCO₂ is greater than 40 mmHg (compared to set point) an error signal is sent to the CPU 110. The CPU 110 can then determine the appropriate corrective measure based on the deviation of the monitored parameter from the established set point, for example.

The apparatus 102 includes a device 112 adapted to output an alarm warning 114 as directed by the CPU 110 when the CPU 110 determines that a corrective measure for the patient 104 is required. The alarm warning 114 is depicted in FIG. 1 as an audible warning such as from the speaker of a sound system for example. It should be understood, however, that the warning of the present invention could embody different forms of warnings such as a visual warning, for example. As described below, the alarm warning 114 of the present invention has a characteristic signature that serves to distinguish the warning from other warnings so that a caregiver of the care setting 100, such as a nurse or health care technician, can rapidly identify the appropriate corrective measure that is needed for a particular patient.

The alarm warning 114 of the present invention has a signature that functions to distinguish the alarm warning 114 from other alarm warnings. In this manner the alarm warning 114 can be adapted to characterize the severity or level of emergency (e.g., yellow, orange, red code levels) and the associated corrective measure that is appropriate to the given level of emergency. For example, the most emergent response, “code red,” might be associated with a complete breakdown of respiratory, cardiac and thermal physiological parameters, thus requiring the most immediate and intense response by the caregiver, or in the case of the closed loop system, the maximum system response. In addition, because most alarms are very similar in sound, the signature feature of the alarm warning 114 is preferably adapted so that the caregiver knows the identity of patient 104 (i.e., identifies which one of a plurality of patients in the health care setting 100 requires help).

According to one preferred embodiment, the alarm warning 114 is audible in the form of music and the signature characteristic distinguishing the alarm warning 114 is based on a property of the music (e.g., genre, tempo, loudness, pitch, etc.) Thus, a digital playback “tune” can be associated with a particular level of urgency, as well as indicate which patient needs help. The caregivers of the health care setting 100 are educated to the “tunes” associated with each patient of the setting 100 as well as the levels of urgency for the patients. For example, a rapid intense version of a classical song might indicate extreme abnormalities in patient “A”, whereas, a traditional up-scale traditional song might indicate extreme abnormalities in patient “B.” The songs and tones are programmed for each level of need (e.g., red, orange, yellow) and for particular patients when more than one patient is involved. Preferably, the music is stored for retrieval by the apparatus 102 on a music storage medium (e.g., mp 3 or other format). Furthermore, the songs and tones would be programmed for each level of need (red, orange, yellow) and individual patients when more than one patient is involved.

As described above, the depicted patient 104 is located within a controlled environment. As should be understood, it is desirable for such a patient that the controlled environment remain as undisturbed as possible. For patients suffering from conditions such as sleep apnea, it is common to apply a waking stimulus to the patient as the appropriate corrective measure during an episode. Traditionally, the waking stimulus would take the form of a nudge physically applied to the patient by a nurse or other caregiver. However, the application of such a corrective measure in the depicted care setting 100 would require that the controlled environment 106 be breached in order to provide access for the caregiver to the patient. The apparatus includes a stimulus device 116 preferably located within the controlled environment 106 adjacent the patient 104 as shown and adapted to generate a waking stimulus. The waking stimulus could embody any of a variety of forms including audio, visual (e.g., flashing light), physical (e.g., vibrating bed or pillow), and scent. The stimulus device 116 is controlled by a driver 118 in response to a signal from the CPU 110. According to one preferred embodiment, the control of the stimulus device 116 by CPU 110 is an adaptive process in that the CPU 110 stores each signal and integrates the errors so that the gain (proportional, differential and integrative feedback response) of the driver 118 is greater depending on the frequency and magnitude of each subsequent error. In addition, the driver 118 will preferably cascade the other feedback signals (oxygen, motion) to proportionally adapt the driver stimulus in a graded fashion. Alternatively, the apparatus could be adapted to provide for an “open-loop” process in which a caregiver has some control over the operation of the driver 118 independently from the CPU 110.

The morbidity and mortality from infections acquired in health care settings is enormous (i.e., estimated at greater than 2 million infants, children and adults per year). Such infections increase the costs of intensive care, prolonging hospitalization by several weeks, and are responsible for almost 50 percent of the deaths that occur beyond two weeks of age. Hand-washing remains the simplest and most effective method for preventing transmission of infectious agents from clinicians to patients. However, in the presence of emergent problems like cardiac/respiratory events, caregivers will break the hand-washing rule and the thermal environment (which takes significant time to recover) in order to stimulate cardiac and respiratory function. Thus, the features of associated with the present invention of (1) identifying the level of emergency, (2) identifying the patient involved, and (3) potentially automatically stimulating the patient during an event or allowing the caregiver the option of stimulating the patient remotely without breaking the environment desirably limit the risk of infection in the health care setting 100.

It should be understood, however, that it is not a requirement of the invention that the corrective measure be applied automatically (e.g., by driver 118 and stimulus device 116 as described above). Instead, the corrective measure could be applied manually by a caregiver of the care setting in response to the alarm warning 114 output by the warning device 112.

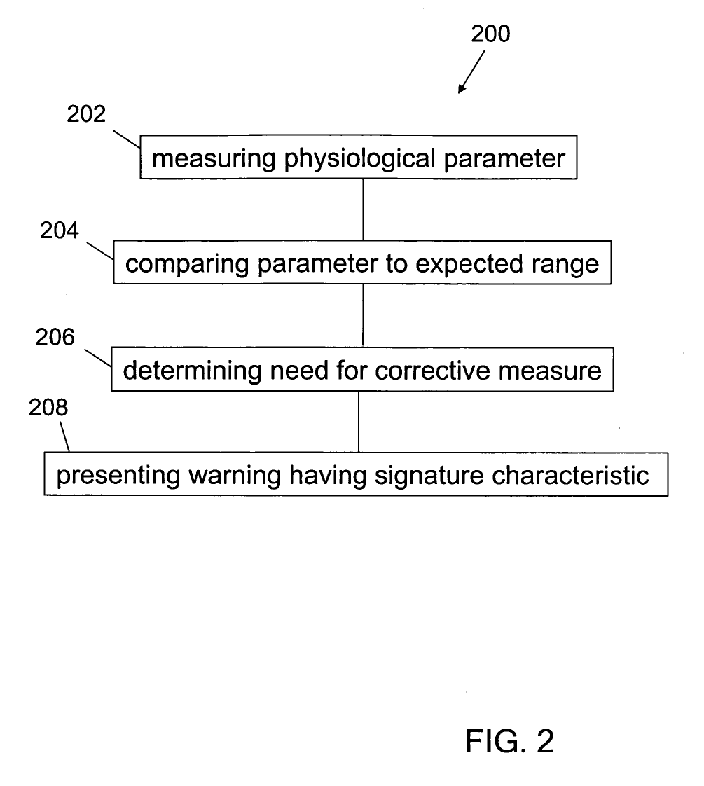

Referring to FIG. 2, the flowchart illustrates a method 200 for maintaining homeostasis for a patient in an intensive care, hospital care or home care setting according to an exemplary embodiment of the invention. The method 200 includes steps 202 and 204 of respectively monitoring the patient for at least one physiological parameter and comparing the monitored parameter against an expected parameter range for the patient. According to step 206 of method 200, a patient condition requiring the application of a corrective measure is determined based on the difference between the monitored parameter and the expected parameter range. Consequently, in step 208 of method 200, a warning is presented to indicate that a corrective measure for the patient is required. According to the claimed method, the warning has a signature characteristic distinguishing the warning from other warnings.

According to one embodiment of method 200, the patient suffers from sleep apnea and the monitored physiological parameter includes one or more of a respiratory parameter, a cardiac parameter and a thermal parameter.

According to a presently preferred embodiment of the invention, the warning presented in step 208 of method 200 is an audible warning. The audible warning may be musical in nature and the signature characteristic distinguishing the warning from other warnings based on the genre of the music. Alternatively, the distinguishing characteristic of the music may be based on the tempo of the music, the pitch of the music or the loudness of the music.

The present invention, however, is not limited to the use of audible warnings in step 208 of method 200. The warning presented in step 208 may embody other forms such as visual warnings for example. It is contemplated that the distinguishing characteristic associated with the visual warning could be based on color, brightness, flashing speed, etc.

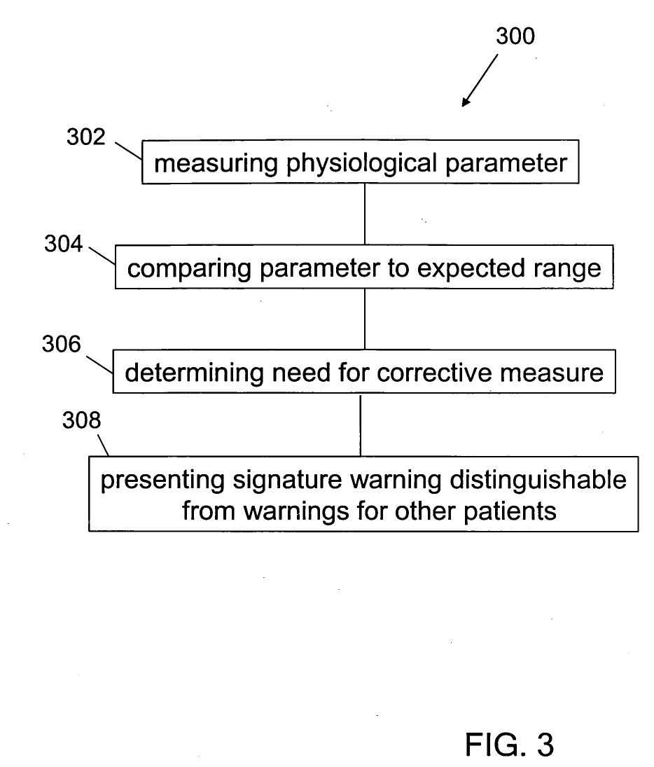

Referring to FIG. 3, there is illustrated in the flowchart a method 300 for maintaining homeostasis for a patient in an intensive care, hospital care or home care setting according to an exemplary embodiment of the invention. The patient of method 300 is one of a plurality of patients at the care setting. The method 300 includes the step 302 of monitoring the patient for a physiological parameter such as a respiratory, cardiac or thermal parameter for example. In step 304, the monitored parameter is compared to an expected range for the physiological parameter. It is then determined in step 306, based on the deviation of the measured parameter from the expected parameter range, that a patient condition exists requiring the application of a corrective measure to the patient. A warning is then presented in step 308, to alert a caregiver for example, indicating the existence of the patient condition requiring a corrective measure. The alarm warning of step 308 has a signature characteristic so as to distinguish the alarm warning from warnings associated with the other patients of the care facility.

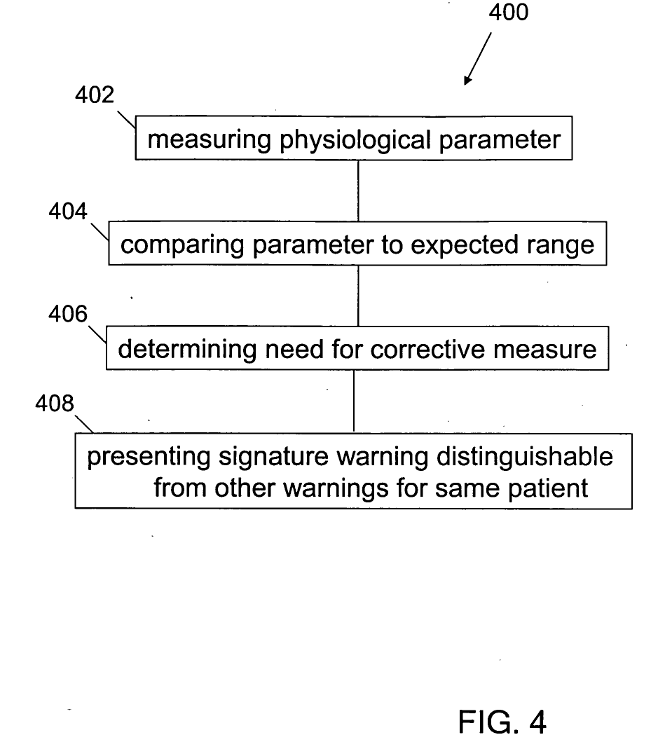

Referring to FIG. 4, there is illustrated in the flowchart a method 400 for maintaining homeostasis for a patient in an intensive care, hospital care or home care setting according to an exemplary embodiment of the invention. For the patient of method 400, there is more than one warning associated with that patient that could be presented to a caregiver of the facility. The plurality of warnings associated with the patient could indicate differing levels of severity associated with a given patient condition (e.g., sleep apnea). Alternatively, the plurality of warnings could indicate differing types of patient conditions suffered by the same patient. The method 400 includes steps 402 and 404 of respectively monitoring the patient for at least one physiological parameter and comparing the monitored parameter against an expected parameter range for the patient. According to step 406 of method 400, a patient condition requiring the application of a corrective measure is determined based on the deviation of the monitored parameter from the expected range. In step 408, the warning having the signature characteristic is presented by which the caregiver is able to distinguish the warning associated with the given condition from the other warnings associated with the same patient.

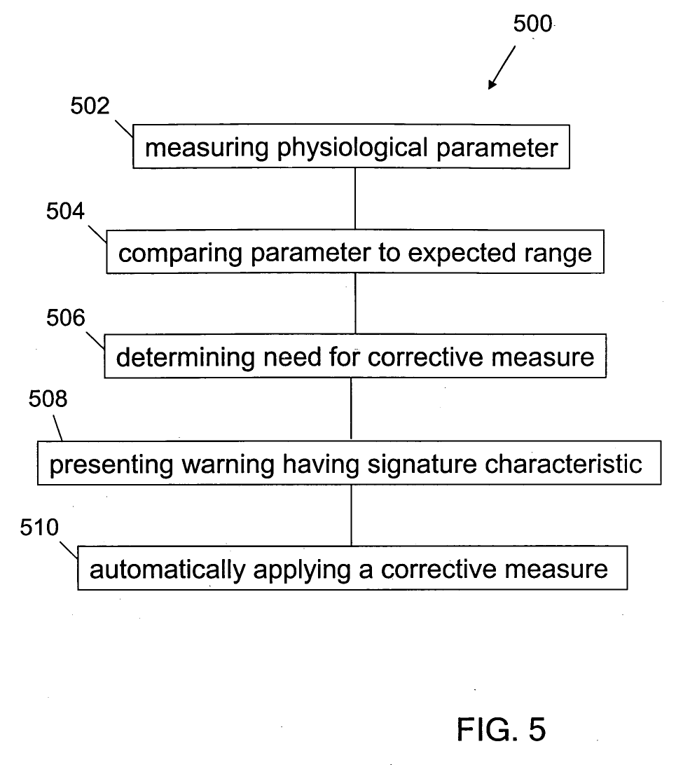

Referring to FIG. 5, there is illustrated in the flowchart a method 500 for maintaining homeostasis for a patient in an intensive care, hospital care or home care setting according to an exemplary embodiment of the invention. The method 500 includes step 502 of monitoring a patient for at least one physiological parameter and step 504 of comparing the monitored parameter against an expected parameter range. In step 506, it is determined, based on the deviation of the monitored parameter from the expected range that a patient condition exists requiring the application of a corrective measure. In step 508, a warning is presented having a signature characteristic by which the warning is distinguishable from other warnings. Method 500 includes the step 510 of automatically applying the required corrective measure to the patient. Method 500, therefore, eliminates the need for action on the part of a caregiver to apply the appropriate corrective measure to the patient.

The foregoing describes the invention in terms of embodiments foreseen by the inventor for which an enabling description was available, notwithstanding that insubstantial modifications of the invention, not presently foreseen, may nonetheless represent equivalents thereto.

What is claimed:

1. A method for maintaining homeostasis for a patient in an intensive care, hospital care or home care setting comprising:
monitoring the patient for at least one physiological parameter;
comparing the monitored physiological parameter against an expected parameter range for the patient;
determining when a difference between the monitored physiological parameter and the expected parameter range presents a patient condition requiring a corrective measure; and
presenting an alarm warning indicating that the patient requires a corrective measure, the alarm warning being one of a plurality of alarm warnings and having a signature characteristic distinguishing the alarm warning from the other alarm warnings.

2. The method of claim 1, wherein the alarm warning is an audible warning.

3. The method of claim 2, wherein the audible warning is musical and wherein the signature characteristic is based on a musical property.

4. The method of claim 3, wherein the signature characteristic is based on musical genre.

5. The method of claim 3, wherein the signature characteristic is based on tempo.

6. The method of claim 2, wherein the signature characteristic is based on pitch.

7. The method of claim 2, wherein the signature characteristic is based on loudness.

8. The method of claim 1 further comprising the step of applying a corrective measure to the patient.

9. The method of claim 1, wherein the patient is one of a plurality of patients of the care setting and wherein the alarm warning associated with the patient is distinguishable from the alarm warnings associated with other patients of the care setting.

10. The method of claim 1, wherein the alarm warning is one of a plurality of alarm warnings associated with a particular patient identifying varying levels of severity associated with the patient condition.

11. The method of claim 1, wherein the alarm warning is one of a plurality of alarm warnings associated with a particular patient identifying patient conditions of differing type suffered by the same patient.

12. The method of claim 1, wherein the patient suffers from sleep apnea and wherein the at least one physiological parameter includes one or more of a respiratory parameter, a cardiac parameter and a thermal parameter.

13. The method of claim 1, wherein the alarm warning is a visual warning.

14. A method for maintaining homeostasis for a patient in an intensive care, hospital care or home care setting having a plurality of patients comprising:
monitoring the patient for at least one physiological parameter;
comparing the monitored physiological parameter against an expected parameter range for the patient;
determining when a difference between the monitored physiological parameter and the expected parameter range presents a patient condition requiring a corrective measure; and
presenting an alarm warning indicating that the patient requires a corrective measure, the alarm warning having a signature characteristic distinguishing the alarm warning associated with the patient from alarm warnings associated with other patients at the setting.

15. The method of claim 14, wherein the alarm warning is audible.
